(19) United States
(12) Patent Application Publication
(10) Pub. No.: US 2007/0049813 A1
(43) Pub. Date: Mar. 1, 2007

(54) Optical sensor for sports equipment

(76) Inventor: David Blouin, Thetford Mines (CA)

Correspondence Address: OGILVY RENAULT LLP, 1981 MCGILL COLLEGE AVENUE, SUITE 1600, MONTREAL, QC, H3A2Y3, CA

(21) Appl. No.: 11/210,893

(22) Filed: Aug. 25, 2005

(51) Int. Cl. A61B 5/00; A61B 5/02

(52) U.S. Cl. 600/340; 600/310; 600/344; 600/502

(57) ABSTRACT

The invention provides a method and an apparatus to monitor a person's vital signs such as, for example, heart rate and blood oxygen level, using non-invasive optical sensors. The invention provides an optical sensor for integration to sports equipment, the sensor comprising: a lighting system for mounting to the sports equipment, the lighting system for providing a light intensity for scattering by a user's skin; and a detection system for mounting to the sports equipment, for detecting the scattered light and for providing a corresponding signal, the corresponding signal being associated to a vital sign of the user. Advantageously, the optical sensor comprises a plurality of near-infrared sources, of red sources and of detectors that are distributed on a surface so as to form a sensing array. Advantageously, the optical sensor is integrated to the handle of sports equipment.

### FIELD OF THE INVENTION

The invention relates to heart beat and oxygen level monitoring systems based on optical sensors that are integrated to a piece of equipment. In particular, the invention relates to heart beat and oxygen level monitoring systems optical sensors integrable to a handle forming part of sports equipment.

### BACKGROUND OF THE ART

Monitoring of a person's heart beat rate has been performed traditionally by measuring the electrocardiogram via electrodes attached to the abdomen. Although this technique has proven to be very efficient, the technique involves the use of electrodes that must be in contact with the skin and thus often embarrass the movements of the person exercising.

The monitoring of a person's heart beat rate may also be performed using optical methods. Optical methods offer the advantage of being non-invasive techniques since only light must interact with the skin and therefore no material contact of a detector to the person's skin is necessary to obtain a measurement. Furthermore, the measurement does not need to be performed near the heart: other parts of the body may be probed by an optical sensor to monitor heart rate beat. However, this technique produces signals that are hard to detect and, for this reason, any movement of the probed skin relative to the optical sensor may be fatal to the measurement. As a result, optical sensors are in practice attached to a body member of the person in order correctly monitor the heart beat rate of a person especially when this person is moving. This again often embarrasses the movements of the person exercising.

There is a need for an improved optical heart beat monitoring system that overcomes the above drawbacks.

### SUMMARY

The invention provides a method and an apparatus to monitor a person's vital signs such as, for example, heart rate and blood oxygen level, using non-invasive optical sensors. The invention provides an optical heart beat rate monitoring sensor and a blood oxygen level monitoring sensor that can be integrated into a piece of equipment that is susceptible to be into contact with a body part of the person using the equipment. For example, it can be integrated to the handlebar of a training machine such as a treadmill, an elliptical machine, a stationary bike or any similar type of equipment. In those kinds of applications, the heart rate and blood oximetry information are obtained by analyzing blood flow changes in the palm of the hands of the subject.

Therefore, in accordance with the present invention, there is provided an optical sensor for integration to sports equipment, the sensor comprising: a lighting system for mounting to the sports equipment, the lighting system for providing a light intensity for scattering by a user's skin; and a detection system for mounting to the sports equipment, for detecting the scattered light and for providing a corresponding signal, the corresponding signal being associated to a vital sign of the user. The optical sensor further comprises a support to which is mounted at least one of the lighting system and the detection system. Advantageously, the sports equipment comprises a handle. Advantageously, the lighting system comprises a light source emitting in the near-infrared. Advantageously, the lighting system further comprises a light source emitting in the red portion of the visible spectrum. Advantageously, the optical sensor comprises a plurality of near-infrared sources, of red sources and of detectors that are distributed on a surface so as to form a sensing array.

The invention also provides a handle for optically sensing a vital sign of a person, the handle comprising: a lighting system integrated to the handle, the lighting system for providing a light intensity for scattering by the person's skin; and a detection system for detecting the scattered light and for providing a corresponding signal, the detection system being integrated to the handle, the corresponding signal being associated to the vital sign. Advantageously, the handle is forming part of sports equipment. Advantageously, the lighting system comprises a light source emitting in the near-infrared. Advantageously, the lighting system further comprises a light source emitting in the red portion of the visible spectrum. Advantageously, the handle comprises a plurality of near-infrared sources, of red sources and of detectors that are distributed on a surface so as to form a sensing array.

The invention also provides a method for monitoring a vital sign of a user of sports equipment, the method comprising: emitting a light intensity using a lighting system integrated to the equipment; generating a scattered light intensity by contacting the equipment with a part of the user's body; and detecting the scattered light intensity using a detection system integrated to the equipment and providing a corresponding signal being associated to the vital sign. Advantageously, the method comprises emitting near-infrared intensity. Advantageously, the method comprises modulating the light intensity as a function of time. Advantageously, the method further comprises emitting a light intensity in the red portion of the visible spectrum. The method also comprises emitting a distributed light intensity pattern so as to provide a regular distribution of light intensity as a function of a surface of a portion of the equipment. The method also comprises detecting the scattered light intensity at a plurality of emplacements on the surface of the portion of the equipment, so as to provide a distributed detection of scattered light as a function of the surface. The method further comprises determining from the corresponding signal at least one of a heart beat rate and a blood oxygen level. Advantageously, the heart beat rate and a blood oxygen determination comprises the step of recognizing a waveform shape in the corresponding signal. Advantageously, the heart beat rate and a blood oxygen determination comprises the step of recognizing a waveform repetition rate in the corresponding signal. Advantageously, the heart beat rate and a blood oxygen determination comprises the step of recognizing waveform amplitude in the corresponding signal.

### DESCRIPTION OF THE DRAWINGS

In order that the invention may be readily understood, embodiments of the invention are illustrated by way of example in the accompanying drawings.

FIG. 1 is a fragmented perspective view of a handle equipped with an optical heart beat rate monitoring sensor comprising one detector and one light emitter, in accordance with an embodiment of the present invention;

FIG. 2 is a fragmented perspective view of a handle equipped with an optical heart beat rate monitoring sensor comprising one detector and two light emitters, in accordance with another embodiment of the present invention;

FIG. 3 is a graph representing the light emitters timing of the optical heart beat rate monitoring sensor of FIG. 2;

FIG. 4 is a fragmented perspective view of a handle equipped with an optical heart beat rate monitoring sensor comprising an array of detectors and light emitters, in accordance with yet another embodiment of the present invention;

FIG. 5 is a graph representing various sports equipment equipped with one of the sensors described above: a) a tennis racquet, b) a training machine, c) a bicycle, and d) a driver's wheel;

FIG. 6a to 6c are fragmented cross section views of the handle of FIG. 2, the handle being equipped with various embodiments of the optical heart beat rate monitoring sensor: a) the sensor is embedded in the handle; b) the sensor is partially inserted in the handle; and c) the sensor comprises optical fibers to relay light and scattered light between light emitters and detector; and

FIG. 7 is a fragmented perspective view of a handle equipped with an optical heart beat rate monitoring sensor comprising one detector and two light emitters, in accordance with yet another embodiment of the present invention.

### DETAILED DESCRIPTION

In the following description of the embodiments, references to the accompanying drawings are by way of illustration of an example by which the invention may be practiced. It will be understood that other embodiments may be made without departing from the scope of the invention disclosed.

The present invention provides a method and an apparatus to monitor a person's vital signs such as, for example, heart rate and blood oxygen level, using non-invasive optical sensors. The invention provides an optical heart beat rate monitoring sensor and a blood oxygen level monitoring sensor that can be integrated into a piece of equipment that is susceptible to be into contact with a body part of the person using the equipment. For example, it can be integrated to the handlebar of a training machine such as a treadmill, an elliptical machine, a stationary bike or any similar type of equipment. In those kinds of applications, the heart rate and blood oximetry information are obtained by analyzing blood flow changes in the palm of the hands of the subject.

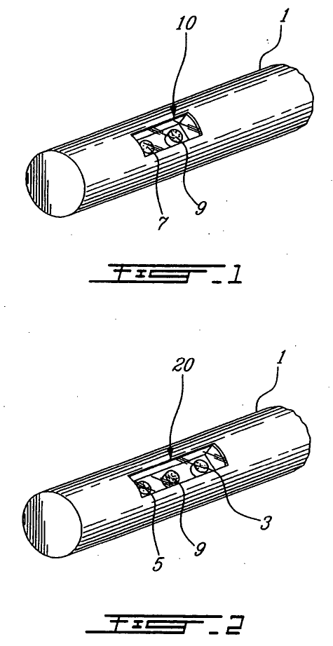

Now referring to FIG. 1, an optical heart beat rate monitoring sensor 10 will be described. In accordance with an embodiment of the present invention, the optical heart beat rate monitoring sensor 10 is integrated into the handle 1 of sports equipment. The sensor 10 comprises a light emitter 7 and detector 9. The light emitter 7 typically emits in the near-infrared part of the light spectrum. It can be, for example, a LED emitting at 950 nm, although other type of sources may also be used. Also, emitter 7 can be the output of an optical fiber (or other kind of light conduit) that is linked, at the other end, to a light source, and that relays the light from the light source to the output of the optical fiber. When a user is gripping handle 1, the palm of the user will absorb and scatter back towards the detector 9 part of the light emitted by the light emitter 7. The intensity of the light scattered by the palm is influenced by the heart beat rate of the user due to the changes in the blood flow and therefore, the intensity of the scattered light, has measured by the detector 9, will be modulated by the heart beat. The detector 9 will thus measure a modulated signal, send this modulated signal to an analysis unit (not shown), enabling the monitoring of the heart beat rate of the user. The detector 9 can be, for example, a photodetector sensitive to near-infrared. It can also be the input of an optical fiber which is linked to a remote photodetector, so as to relay the scattered light to the remote photodetector.

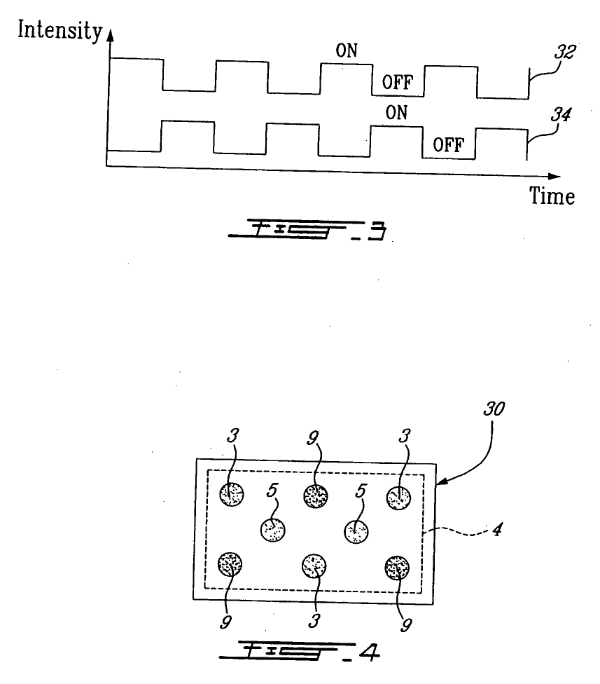

Now referring to FIG. 2, an optical heart beat rate monitoring sensor 20 will be described. In accordance with another embodiment of the present invention, the optical heart beat rate monitoring sensor 20 is integrated into the handle 1 of sports equipment, and provides oximetry of the blood of the user. This is illustrated in FIG. 2. Oximetry enables to determine the oxygen saturation of the blood of the user. Oximetry is based on the fact that the optical property of blood in the near-infrared spectrum and in the red portion of the visible spectrum depends on the amount of oxygen in blood. Sensor 20 is composed of a detector 9, a near-infrared light emitter 5 and a red light emitter 3. The red light emitter can be, for example, a LED emitting around 660 nm. As was the case with sensor 10, the palm of the user will absorb and scatter back towards the detector 9 part of the light emitted by the light emitters 3 and 5. Detector 9 is sensitive enough to detect the near-infrared and visible red lights scattered by the user's palm. The intensity of the near-infrared and visible red scattered lights by the palm is influenced by the heart beat rate of the user due to the changes in the blood flow and is also influenced by the amount of oxygen in the blood. Therefore, the intensity of the scattered light, as measured by the detector 9, will be modulated by the heart beat and the amount of oxygen. The detector 9 will thus measure a modulated signal, send this modulated signal to an analysis unit (not shown), enabling the monitoring of the heart beat rate of the user and the monitoring of the amount of oxygen in his blood.

In order to discriminate between the near-infrared scattered light and the red scattered light, near-infrared 5 and red 3 emitters are alternatively turned on, to have, at a given time, only one light emitter emitting (either near-infrared 32 or red 34) and only either near-infrared or red scattered light. This concept is illustrated on FIG. 3. This particular timing allows the system to independently detect and analyze near-infrared and red wavelengths dynamic light absorption by the blood flow and the static light absorption by the subject's skin and flesh. Knowing this difference in reflection is required to calculate blood oxygen level (pulse oximetry), if required.

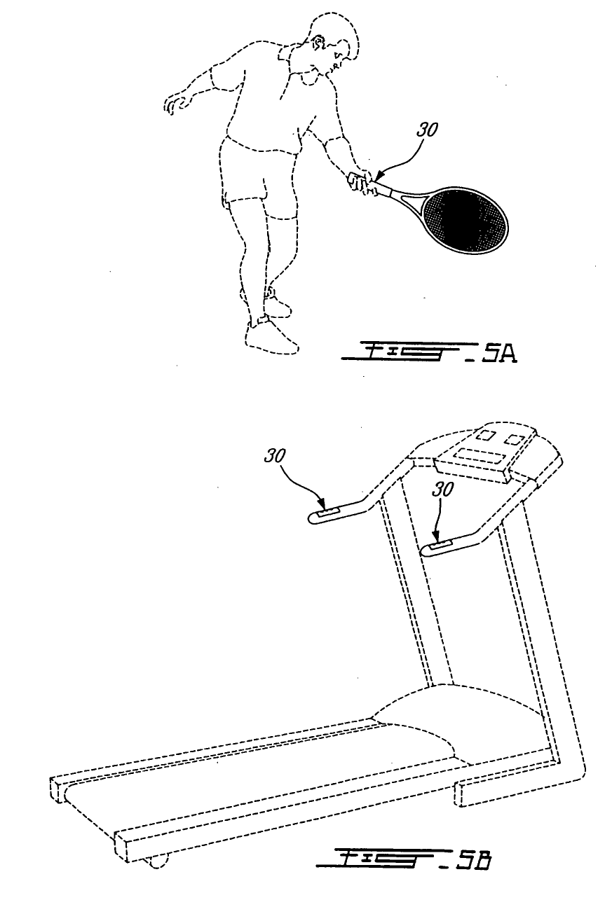

One major challenge of measuring the heart rate beat of the user is posed by the fact that the user's hands are constantly moving along with the subject's body, producing transient blood flow fluctuations, which in turn produce erroneous readings. In accordance with another embodiment of the present invention, an optical heart beat rate monitoring sensor 30, integrated into the handle 1 of sports equipment and being less sensitive to movements of the user's hand, is provided. The sensor 30 is illustrated in FIG. 4. As it was the case for sensor 20, sensor 30 allows monitoring the heart rate beat of the user and the amount of oxygen in user's blood.

Sensor 30 is composed of a multitude of red light emitters 3, near-infrared light emitters 5 and light detectors 9 disposed to form a sensing array 4 of interlaced light emitters and detectors. This disposition of the light emitters and detectors provides an even light distribution in the palm of the hand of the subject, even if the palm is not perfectly centered above the sensing array 4. Thus, even if the user changes his grip, sensor 30 can still monitor user's heart beat rate. It is contemplated that other disposition of the light emitters and sensors are possible. It is also contemplated to use a plurality of optical fibers as light emitters 3,5, some of them being optically coupled to remote red sources, some of them being optically coupled to remote near-infrared sources, and to use a plurality of other optical fibers optically coupled to remote photodetectors, as detectors 9.

Similarly to sensor 20, the near-infrared 5 and red 3 light emitters are alternatively turned on, to have, at a given time, only near-infrared or red light, and the corresponding scattered light. Thus detectors 9 measure as a function of time either near-infrared scattered light or red scattered light. This allows independent analysis of near-infrared and red wavelength dynamic light absorption by the blood flow and static light absorption by the subject's skin and flesh. Knowing this difference in reflection is required to calculate blood oxygen level (pulse oximetry). Using software correlation techniques, light sensors are sampled at specific moments in the cycle to ensure accurate readings.

Examples of sports equipment having sensors 10, 20 or 30 in their handle are shown in FIG. 5. For example, sensor 10, 20 or 30 can be integrated in a tennis racquet, as shown in a), or in the handles of an exercise machine, as shown in b) or in the handles of a bicycle, as shown in c), or in a driver's wheel, as shown in d).

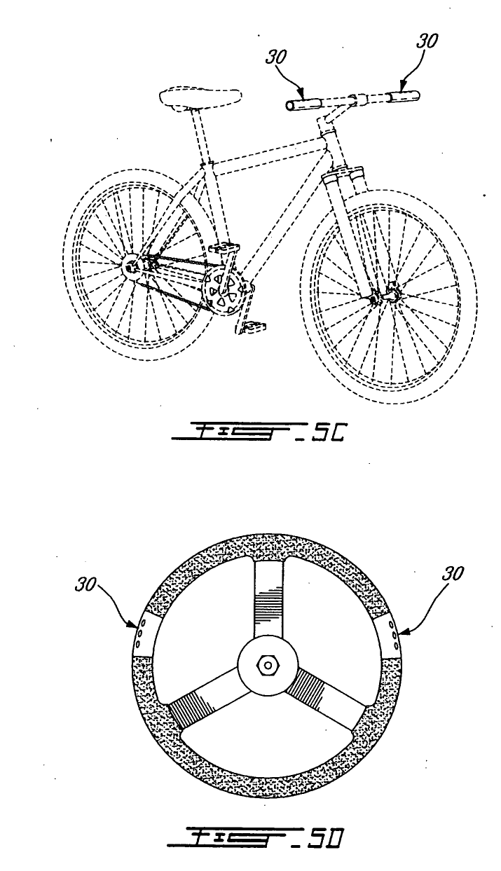

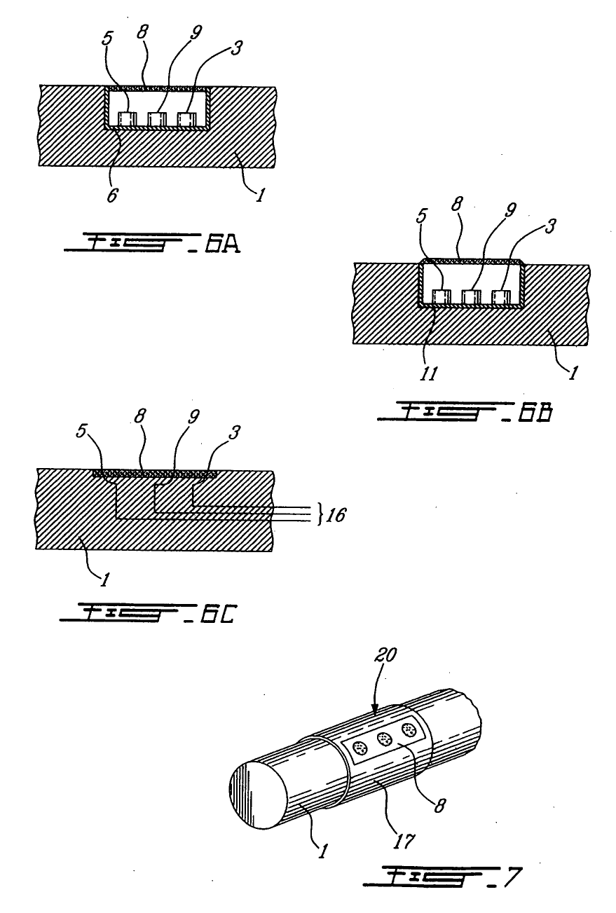

The present invention provides sensors 10, 20, and 30 that are integrable in a piece of equipment to monitor non-invasively a person's vital signs such as heart rate and blood oxygen level. The integration of sensors 10, 20 or 30, can be made to any equipment part that is susceptible to be in contact with a body part of the equipment user. Many ways to integrate sensors 10, 20 or 30 are contemplated by the present invention and some examples are illustrated in FIG. 6a to 6c. In FIG. 6a, sensor 20 is integrated into the handle 1 forming part of the equipment so as to be completely embedded in the handle 1. Light emitters 3,5 and detector 9 are mounted to a support 6 and are protected by a window 8 that transmits the light emitted by the light emitters and the scattered light. The whole configuration provides a compact assembly that is easily integrated in the handle 1. In this configuration, the window is flush to the surface 11 of handle 1 so as to provide a comfortable grip to the user. The window 8 can be a flat piece of appropriate material, such as, for example, a dark tinted polycarbonate panel. It can also be a lens or an array of lenses. In FIG. 6b, sensor 20 has been partially inserted in the handle 1 and the window 8 is slightly protruding the surface 11 of the handle 1. In some circumstances, it may be advantageous to integrate sensors 10, 20 or 30 this way, so as to provide, for example, integrated sensors with a relief that is even more comfortable to the user's grip.

As illustrated in FIG. 6c, sensors 10, 20 and 30 may also be integrated so as to use remote light emitters 7 as well as remote detectors 9. In this particular embodiment, the light emitted by the remote light sources is relayed to the handle 1 by means of an optical fiber cable 16 terminating near the window 8. The optical fiber cable 16 serves also to relay the scattered light to the remote photodetectors.

Many other ways to integrate sensors 10, 20 or 30 to a handle exist and they are all included in the present invention. For example, instead of embedding or partially embedding sensors 10, 20 or 30 in handle 1, sensor 10, 20 or 30, as illustrated in FIG. 7, can be part of a strip 17 that is then affixed to existing handle 1.

The present invention further provides processing the detected signals in order to determine from them the heart rate beat and the amount of oxygen. In one embodiment of the present invention, an analysis unit (not shown) is embedded in the handle and receives the signals detected by the detectors. It then performs all the necessary calculations to provide a heart rate beat monitoring signal and an oximetry signal. In an alternative embodiment, the analysis unit is not part of the handle and detected signals are relayed to the remote analysis unit via wireless signals. As will be obvious for someone skilled in the art, other ways may equally be used to relay the detected signal to the analysis unit. At the reception of the detected signal, the analysis unit computes the heart beat rate monitoring signal and the oximetry monitoring signal of the user. The analysis unit can further comprise a visual display to display as a function of time that heart beat rate and oximetry monitoring signals.

In an embodiment of the present invention, the analysis unit comprises features for filtering and processing the detected signals to provide, after filtering and additional processing, a signal having strong periodic components from the output of each light sensor processing block. These signals are then sent into a statistical analysis subsystem, after appropriate ADC (analog-to-digital) conversion. This subsystem performs several time and amplitude related mathematical analysis on the incoming data streams, to identify at least one valid signal source. As will be obvious for someone skilled in the art, sensing array 4 provides the advantage of multiplying the number of emitters and detectors, therefore giving the possibility of increasing the precision of the provided heart beat rate and oximetry monitoring signals. The analysis unit, by using validity criteria such as, for example, waveform repetition rate, general waveform shape and waveform amplitude of the detected signals as well as the presence of specific signal characteristics and the proper sequence of polarity changes in the waveform, can discard detected signals that are believed to be invalid.

Once a good signal source is found, heart rate calculation is performed on available data, and the result can be sent to a host computer system or, directly displayed on the display. As a by-product of the process, blood oximetry calculations can also be performed, providing information about the oxygen content in the subject's blood flow. Sensors 10, 20 and 30 can further have a controller unit (not shown) for synchronizing the light sources timing, for adjusting the light source output intensity to compensate for adverse lighting condition, for controlling ADC conversion process timing and for managing various functions such as communication with the host computer or with the data display.

Although the present invention has been described hereinabove by way of specific embodiments related to handle forming part of sports equipment, it will be obvious for someone skilled in the art that sensors 10, 20 or 30 can also be integrated to other parts of sports equipment that are susceptible to be in contact with the skin of the user of sports equipment. For example, sensors 10, 20 or 30 can also be integrated to the seat of the equipment.

Although the present invention has been described hereinabove by way of specific embodiments thereof, it can be modified, without departing from the spirit and nature of the subject invention as defined herein. The scope of the invention is therefore intended to be limited solely by the scope of the appended claims.

I claim:

1. An optical sensor for integration to equipment, the sensor comprising:
a lighting system for mounting to the equipment, the lighting system for providing a light intensity for scattering by a user's skin;
a detection system for mounting to the equipment, for detecting the scattered light and for providing a corresponding signal, the corresponding signal being associated to a vital sign of the user, the detection system comprising an analysis unit for determining, from the corresponding signal, said vital sign; and
the analysis unit comprises at least one of a waveform shape recognition algorithm, a waveform repetition rate recognition algorithm and a waveform amplitude recognition algorithm.

2. The sensor of claim 1, further comprising a support to which is mounted at least one of the lighting system and the detection system.

3. The sensor of claim 1, wherein the equipment comprises a handle.

4. The sensor of claim 1, wherein the lighting system comprises a light source emitting in the near-infrared.

5. The sensor of claim 4, wherein the light source comprises a light emitting diode (LED).

6. The sensor of claim 1, wherein the detection system comprises a detector sensitive to the near-infrared.

7. The sensor of claim 1, wherein the lighting system comprises an optical fiber linkable to a remote light source for providing the light intensity.

8. The sensor of claim 1, wherein the detection system comprises an optical fiber linkable to a remote detector for detecting.

9. The sensor of claim 1, wherein the lighting system comprises a first and a second light sources, the first source emitting in the near-infrared and the second source emitting in the red portion of the visible spectrum.

10. The sensor of claim 9, wherein each one of the first and second light sources comprises a light emitting diode (LED).

11. The sensor of claim 9, wherein the detection system comprises a detector sensitive to the near-infrared and to the red portion of the visible spectrum.

12. The sensor of claim 9, wherein the lighting system comprises a control unit for alternatively turning on the first and second light sources.

13. The sensor of claim 1, wherein the lighting system comprises a plurality of near-infrared and red light sources, the near-infrared sources emitting in the near-infrared and the red sources emitting in the red portion of the visible spectrum.

14. The sensor of claim 13, wherein the near-infrared and red sources comprise light emitting diodes (LED).

15. The sensor of claim 13, wherein the detection system comprises a plurality of detectors sensitive to the near-infrared and to the red portion of the visible spectrum.

16. The sensor of claim 13, wherein the near-infrared sources, the red sources and the detectors are distributed on a surface so as to form a sensing array.

17. The sensor of claim 16, wherein the near-infrared sources, the red sources and the detectors are uniformly distributed on the surface.

18. The sensor of claim 13, wherein the light source system comprises a control unit for alternatively turning on the near-infrared and red light sources.

19. The sensor of claim 1, wherein the vital sign comprises at least one of a heart beat rate and an blood oxygen level.

20. The sensor of claim 19, wherein said equipment comprises sports equipment.

21. The sensor of claim 19, further comprising a display unit for displaying one of the heart beat rate and the blood oxygen level.

22. The sensor of claim 19, wherein the detection system comprises means to relay the corresponding signal to the analysis unit.

23. The sensor of claim 22, wherein the means to relay comprises a wireless transmitter.

24. A handle for optically sensing a vital sign of a person; the handle comprising:
a portion for a hand of the person;
a lighting system integrated to the handle, the lighting system for providing a light intensity for scattering by the skin of the person's hand; and
a detection system for detecting the scattered light and providing a corresponding signal, the detection system being integrated to the handle, the corresponding signal being associated to the vital sign.

25. The handle of claim 24, comprising a handle forming part of sports equipment.

26. The handle of claim 24, wherein the lighting system comprises a light source emitting in the near-infrared.

27. The handle of claim 26, wherein the light source comprises a light emitting diode (LED).

28. The handle of claim 24, wherein the detection system comprises a detector sensitive to the near-infrared.

29. The handle of claim 24, wherein the lighting system comprises an optical fiber linkable to a remote light source for providing the light intensity.

30. The handle of claim 24, wherein the detection system comprises an optical fiber linkable to a remote detector for detecting.

31. The handle of claim 24, wherein the lighting system comprises a first and a second light sources, the first source emitting in the near-infrared and the second source emitting in the red portion of the visible spectrum.

32. The handle of claim 31, wherein each one of the first and second light sources comprises a light emitting diode (LED).

33. The handle of claim 31, wherein the detection system comprises a detector sensitive to the near-infrared and to the red portion of the visible spectrum.

34. The handle of claim 31, wherein the lighting system comprises a control unit for alternatively turning on the first and second light sources.

35. The handle of claim 24, wherein the lighting system comprises a plurality of near-infrared and =red light sources, the near-infrared sources emitting in the near-infrared and the red sources emitting in the red portion of the visible spectrum.

36. The handle of claim 35, wherein the near-infrared and red sources comprise light emitting diodes (LED).

37. The handle of claim 35, wherein the detection system comprises a plurality of detectors sensitive to the near-infrared and to the red portion of the visible spectrum.

38. The handle of claim 35, wherein the near-infrared sources, the red sources and the detectors are distributed on a surface so as to form a sensing array.

39. The handle of claim 38, wherein the near-infrared sources, the red sources and the detectors are uniformly distributed on the surface.

40. The handle of claim 35, wherein the light source system comprises a control unit for alternatively turning on the near-infrared and red light sources.

41. The handle of claim 24, wherein the detection system comprises an analysis unit for determining, from the corresponding signal, at least one of the heart beat rate and the blood oxygen level.

42. The handle of claim 41, wherein the analysis unit comprises at least one of a waveform shape recognition algorithm, a waveform repetition rate recognition algorithm and a waveform amplitude recognition.

43. The handle of claim 41, further comprising a display unit for displaying one of the heart beat rate and the blood oxygen level.

44. The handle of claim 41, wherein the detection system comprises means to relay the corresponding signal to the analysis unit.

45. The handle of claim 44, wherein the means to relay comprises a wireless transmitter.

46. A method for monitoring a vital sign of a user of equipment, the method comprising:
emitting a light intensity using a lighting system integrated to the equipment;
generating a scattered light intensity by contacting the equipment with a part of the user's body; and
detecting the scattered light intensity using a detection system integrated to the equipment and determining a corresponding signal being associated to the vital sign; said determining comprises at least one of recognizing a waveform shape in the corresponding signal; recognizing a waveform repetition rate in the corresponding signal; and recognizing a waveform amplitude in the corresponding signal.

47. The method as claimed in claim 46, wherein emitting the light intensity comprises emitting a near-infrared intensity.

48. The method as claimed in claim 46, wherein emitting the light intensity comprises modulating the light intensity as a function of time.

49. The method of claim 46, wherein emitting the light intensity comprises emitting a near-infrared intensity and a visible red intensity.

50. The method as claimed in claim 49, wherein emitting the light intensity comprises emitting the near-infrared intensity and the visible red intensity alternatively as a function of time.

51. The method of claim 46, wherein emitting the light intensity comprises emitting a distributed light intensity pattern so as to provide a regular distribution of light intensity as a function of a surface of a portion of the equipment.

52. The method as claimed in claim 51, wherein detecting the scattered light intensity comprises detecting the scattered light intensity at a plurality of emplacements on the surface of the portion of the equipment, so as to provide a distributed detection of scattered light as a function of the surface.

53. The method as claimed in claim 46, further comprising determining from the corresponding signal at least one of a heart beat rate and a blood oxygen level.
